(19) United States
(12) Patent Application Publication
(10) Pub. No.: US 2013/0338390 A1
(43) Pub. Date: Dec. 19, 2013

(54) SYNTHESIS OF RESVERATROL-BASED COMPOUNDS

(71) Applicant: Scott Alan Snyder, Dobbs Ferry, NY (US)

(72) Inventor: Scott Alan Snyder, Dobbs Ferry, NY (US)

(73) Assignee: The Trustees of Columbia University in the City of New York, New York, NY (US)

(21) Appl. No.: 13/810,093

(22) PCT Filed: Jul. 21, 2011
(86) PCT No.: PCT/US11/44806

§ 371 Date: Aug. 23, 2013

Related U.S. Application Data
(60) Provisional application No. 61/400,111, filed on Jul. 21, 2010.

(51) Int. Cl. C07F 9/06; C07C 39/17; C07C 49/747; C07C 69/738; C07C 323/17

(52) U.S. Cl. CPC C07F 9/06; C07C 69/738; C07C 323/17; C07C 49/747; C07C 39/17
USPC 558/198; 560/176; 568/49; 568/327; 568/719

(57) ABSTRACT

A compound having the structure:

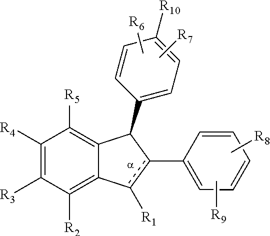

wherein
bond α is present or absent,
   wherein when bond α is absent, then R₁ is ═O or S(CH₂)₁₋₃-phenyl and when bond α is present, then R₁ is H,
      wherein the phenyl is substituted or unsubstituted;
R₂, R₃, R₄, R₅, R₆, R₇, R₈, R₉ and R₁₀ are independently H or OR₁₁
   wherein each occurrence of R₁₁ is independently H, methyl, substituted or
   unsubstituted alkyl, substituted or unsubstituted aryl, phosphate, sulfate, sulfonic ester, or ester;
when α is absent and R₁ is ═O, then R₁₀ is other than OH; and
when α is absent and R₁ is S(CH₂)₁₋₃-phenyl, then R₁₀ is other than OCH₃,
   or a salt thereof.

This application claims priority of U.S. Provisional Application No. 61/400,111, filed Jul. 21, 2010, the contents of which are hereby incorporated by reference.

Throughout this application, various publications are referenced by citation in parentheses. The disclosures of these publications in their entireties are hereby incorporated by reference into this application in order to more fully describe the state of the art as known to those skilled therein as of the date of the invention described and claimed herein.

### BACKGROUND

The past decade has witnessed tremendous interest in the relatively small natural product resveratrol (1, FIG. 1) based primarily on its possession of a promising and selective array of in vitro and in viva activity against a collection of disease states, including inflammation, heart disease, aging, and cancer [1]. In fact, its truly unique biochemical profile, coupled with its relatively high concentration in red wine (˜100 mM) and near absence in white varietals and grape juice, has led to the popularly held notion that resveratrol is the main protagonist for the so-called “French paradox” [2]. Amazingly, however, virtually no effort has been devoted to the large family of resveratrol-based oligomers (such as 2-8) [3] produced combinatorially by plants throughout the world in response to environmental stress, compounds which initial screening suggest should have unique, if not superior, activity profiles to resveratrol itself [4].

### SUMMARY OF THE INVENTION

In one embodiment, this invention provides a compound having the structure

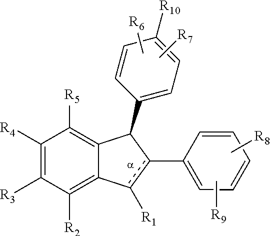

wherein
bond α is present or absent,
   wherein when bond α is absent, then R₁ is ═O or S(CH₂)₁₋₃-phenyl and
   when bond α is present, then R₁ is H,
      wherein the phenyl is substituted or unsubstituted;
R₂, R₃, R₄, R₅, R₆, R₇, R₈, R₉ and R₁₀ are independently H or OR₁₁
   wherein each occurrence of R₁₁ is independently H, methyl, substituted or
   unsubstituted alkyl, substituted or unsubstituted aryl, phosphate, sulfate, sulfonic ester, or ester;
when α is absent and R₁ is ═O, then R₁₀ is other than OH; and
when α is absent and R₁ is S(CH₂)₁₋₃-phenyl, then R₁₀ is other than OCH₃, or a salt thereof.

### BRIEF DESCRIPTION OF THE FIGURES

FIG. 1: Selected examples of polyphenolic natural products presumed to arise from the union of resveratrol monomers.

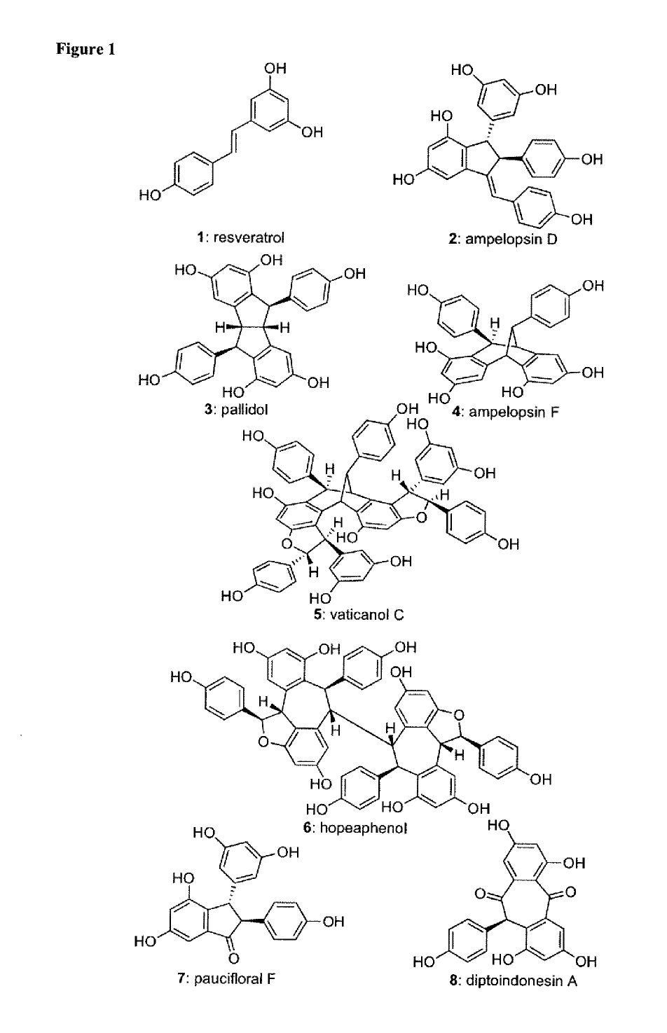

FIG. 2: Synthetic scheme detailing the synthesis of compounds A, C, D, F, G, I, and J.

FIG. 3: Synthetic scheme detailing the synthesis of compounds B, E and H.

### DETAILED DESCRIPTION

This invention provides a compound having the structure

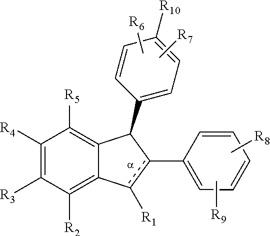

wherein
bond α is present or absent,
   wherein when bond α is absent, then R, is ═O or S(CH₂)₁₋₃-phenyl and
   when bond α is present, then R, is H,
      wherein the phenyl is substituted or unsubstituted;
R₂, R₃, R₄, R₅, R₆, R₇, R₈, R₉ and R₁₀ are independently H or OR₁₁
   wherein each occurrence of R₁₁ is independently H, methyl, substituted or
   unsubstituted alkyl, substituted or unsubstituted heteroalkyl substituted or
   unsubstituted aryl, substituted or unsubstituted aryl, phosphate, sulfate, sulfonic ester, or ester;
when α is absent and R₁ is ═O, then R₁₀ is other than OH; and
when α is absent and R₁ is S(CH₂)₁₋₃-phenyl, then R₁₀ is other than OCH₃,
   or a salt thereof.

In some embodiments, the invention includes the compound wherein in the compound each occurrence of R₁₁ is independently H, CH₃, C(O)CH₃, P(O)(OR₁₂)₂, SO₂OR₁₂, SO₂R₁₃, or C(O)R₁₃,
wherein each occurrence of R₁₂ is independently H, methyl, substituted or unsubstituted alkyl, or substituted or unsubstituted aryl,
wherein each occurrence of R₁₃ is independently H, methyl, substituted or unsubstituted alkyl, or substituted or unsubstituted aryl,
   or a salt thereof.

In some embodiments, the invention includes the compound wherein in the compound
R₃ and R₅ are OR₁₁; and R₂ and R₄ are H,
   or a salt thereof.

In some embodiments, the invention includes the compound

wherein the structure is

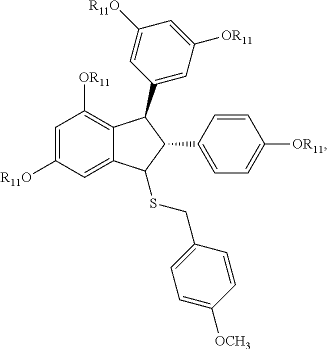

or a salt thereof.

In some embodiments, the invention includes the compound wherein in the compound R₁₁ is H, CH₃, C(O)CH₃, or P(O)(OR₁₂)₂,
wherein R₁₂ is H,
   or a salt thereof.

In some embodiments, the invention includes the compound

wherein the structure is

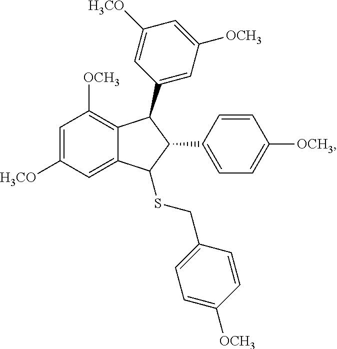 [SMILES: COc1ccc(CSC2c3cc(OC)cc(OC)c3[C@@H](c3cc(OC)cc(OC)c3)[C@@H]2c2ccc(OC)cc2)cc1]

or a salt thereof.

In some embodiments, the invention includes the compound

wherein the structure is

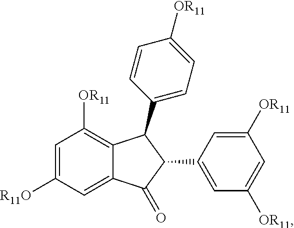

or a salt thereof.

In some embodiments, the invention includes the compound wherein in the compound R₁₁ is CH₃, C(O)CH₃, or P(O)(OR₁₂)₂,
wherein R₁₂ is H,
   or a salt thereof.

In some embodiments, the invention includes the compound

wherein the structure is

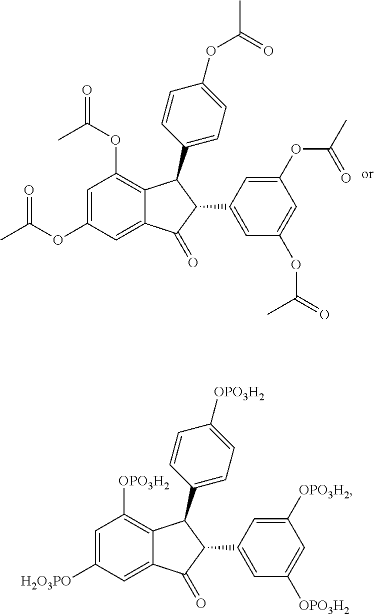

or a salt thereof.

In some embodiments, the invention includes the compound

wherein the structure is

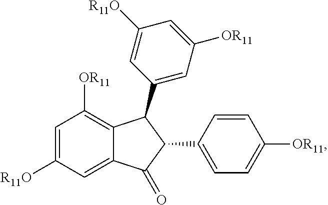

or a salt thereof.

In some embodiments, the invention includes the compound wherein in the compound R₁₁ is H, CH₃, C(O)CH₃, P(O)(OR₁₂)₂,
wherein R₁₂ is H,
   or a salt thereof.

In some embodiments, the invention includes the compound wherein the structure is

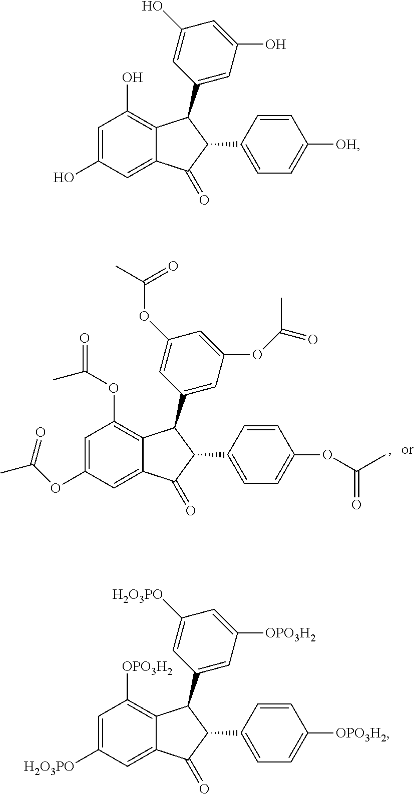

or a salt thereof.

In some embodiments, the invention includes the compound wherein the structure is

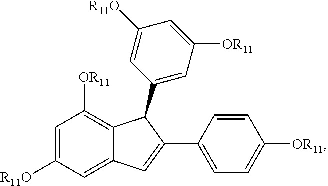

or a salt thereof.

In some embodiments, the invention includes the compound wherein in the compound R₁₁ is H, CH₃, C(O)CH₃, P(O)(OR₁₂)₂,
wherein R₁₂ is H,
   or a salt thereof.

In some embodiments, the invention includes the compound wherein the structure is

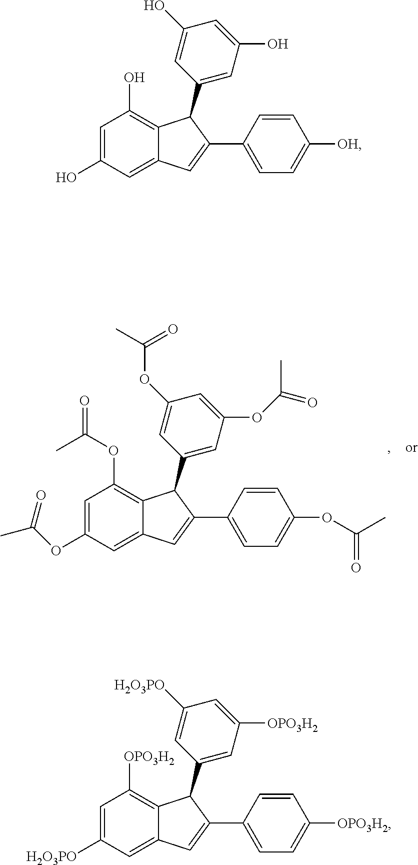

or a salt thereof.

In some embodiments, the invention includes the compound wherein the structure is

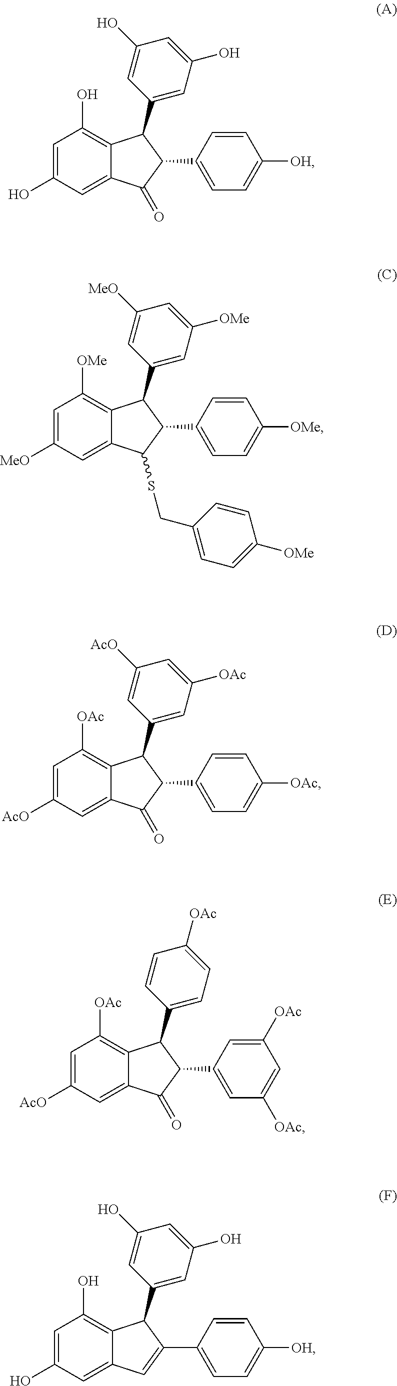 [SMILES: O=C1c2cc(O)cc(O)c2[C@@H](c2cc(O)cc(O)c2)[C@@H]1c1ccc(O)cc1 ; COc1ccc(CSC2c3cc(OC)cc(OC)c3[C@@H](c3cc(OC)cc(OC)c3)[C@@H]2c2ccc(OC)cc2)cc1 ; CC(=O)Oc1ccc([C@@H]2C(=O)c3cc(OC(C)=O)cc(OC(C)=O)c3[C@H]2c2cc(OC(C)=O)cc(OC(C)=O)c2)cc1 ; CC(=O)Oc1ccc([C@@H]2c3c(OC(C)=O)cc(OC(C)=O)cc3C(=O)[C@H]2c2cc(OC(C)=O)cc(OC(C)=O)c2)cc1 ; Oc1ccc(C2=Cc3cc(O)cc(O)c3[C@H]2c2cc(O)cc(O)c2)cc1]

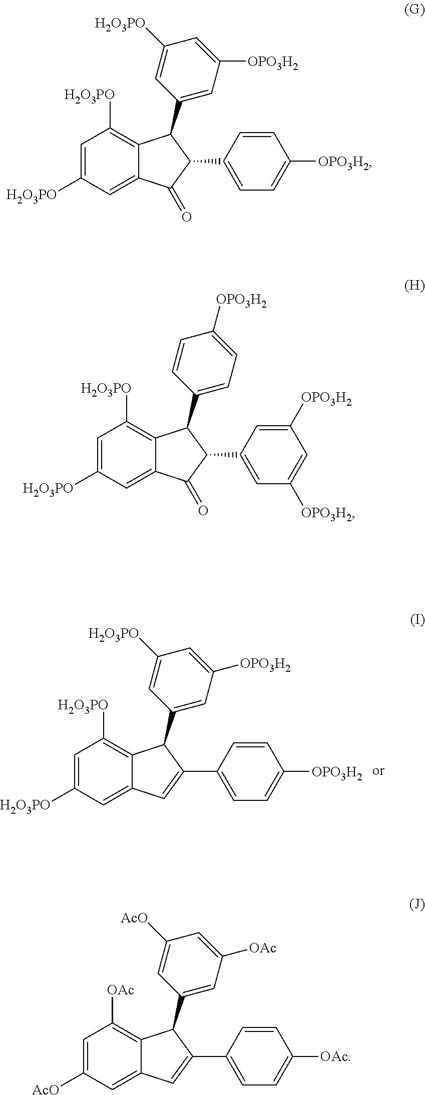

In some embodiments, the invention provides a pharmaceutical composition comprising a compound of the invention and a pharmaceutically acceptable carrier.

In some embodiments, a compound for use in reducing, preventing or inhibiting a fungal infection of a plant or animal comprising contacting the fungus with the compound in an amount effective to reduce, prevent or inhibit the fungal infection.

In some embodiments, a compound for use in inhibiting fungal growth or fungal proliferation comprising contacting the fungus with the compound in an amount effective to inhibit growth or proliferation of the fungus.

In some embodiments, a compound for use in reducing the transmission of ultraviolet light to a surface exposed thereto comprising contacting the surface with the compound in an amount effective to reduce the transmission of ultraviolet light to the surface.

In some embodiments, the ultraviolet light is UV-B.

In some embodiments, the surface is the skin of a subject.

In some embodiments, a compound for use in treating a skin cancer in a subject comprising contacting the skin cancer with the compound in amount effective to treat the skin cancer.

In some embodiments, the subject is a human.

This invention provides for use of one or more of the above compounds in the manufacture of a medicament for inhibiting fungal growth or fungal proliferation in a subject.

This invention provides one or more of the above compounds for use in inhibiting fungal growth or fungal proliferation in a subject.

This invention provides for use of the one or more of the above compositions in the manufacture of a medicament for inhibiting fungal growth or fungal proliferation in a subject.

This invention provides one or more of the above compositions for use in inhibiting fungal growth or fungal proliferation in a subject.

This invention provides a method for reducing the transmission of ultraviolet light to a surface exposed thereto comprising contacting the surface with one or more of the above compounds in amount effective to reduce the transmission of ultraviolet light to the surface.

This invention provides a method for reducing the transmission of ultraviolet light to a surface exposed thereto comprising contacting the surface with one or more of the above compositions in amount effective to reduce the transmission of ultraviolet light to the surface.

This invention provides a method for treating a skin cancer in a subject comprising contacting the skin cancer with one or more of the above compounds in amount effective to treat the skin cancer.

This invention provides a method for treating a skin cancer in a subject comprising contacting the skin cancer with one or more of the above compositions in amount effective to treat the skin cancer.

In some embodiments, the skin cancer is a malignant melanoma or a basal cell carcinoma.

In some embodiments, the subject is a human.

This invention provides for use of one or more of the above compounds in the manufacture of a medicament for treating a skin cancer in a subject.

This invention provides one or more of the above compounds for use in the treatment of a skin cancer in a subject.

This invention provides for use of one or more of the above compositions in the manufacture of a medicament for treating a skin cancer in a subject.

This invention provides one or more of the above compositions for use in the treatment of a skin cancer in a subject.

In the fungicidal and fungal-retarding methods described hereinabove it is understood that the compounds and compositions can act on the fungus itself or on the spores of the fungus to achieve their effect. In addition, the compounds and compositions can act on oomycetes to impair their growth or prevent infection by oomycetes, and methods for doing such are also provided herein. The compounds and compositions may be applied for example, in the case of plants and animals, by spraying of, or dipping/immersion in, the compounds or compositions. Alternatively, they may be applied as pharmaceutical compositions comprising a pharmaceutically acceptable carrier.

In some embodiments, the invention provides a compound free of plant extract.

“Free of plant extract” with regard to a composition as used here means that the composition is absent any amount of resveratrol containing-plant material or resveratrol-based oligomer containing-plant material. Thus only synthetically produced compounds and compositions are free of plant extract. Any compound or compositions isolated from a plant would always contain at least some trace amount of plant material.

A method is provided for reducing the degree of a fungal infection comprising contacting the fungi with a compound described herein an in amount effective to reduce the degree of the fungal infection.

A method is provided for preventing or impairing a fungal infection comprising contacting the fungi with a compound described herein an in amount effective to prevent or impair the fungal infection.

As used herein, “alkyl” includes both branched and straight-chain saturated aliphatic hydrocarbon groups having the specified number of carbon atoms. Thus, C₁-Cₙ as in “C₁-Cₙ alkyl” is defined to include groups having 1, 2, . . . , n-1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, propyl, butyl, pentyl, hexyl, and so on. In an embodiment the alkyl is C₁ (methyl). In an embodiment the alkyl is a C₂-C₇ alkyl. In embodiments the alkyl is a C₁, C₂, C₃, C₄, C₅, C₆, C₇, C₈, C₉, or C₁₀ alkyl.

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “alkenyl” refers to a non-aromatic hydrocarbon radical, straight or branched, containing at least 1 carbon to carbon double bond, and up to the maximum possible number of non-aromatic carbon-carbon double bonds may be present. For example, “C₂-C₆ alkenyl” means an alkenyl radical having 2, 3, 4, 5, or 6 carbon atoms, and up to 1, 2, 3, 4, or 5 carbon-carbon double bonds respectively. Alkenyl groups include ethenyl, propenyl, butenyl and cyclohexenyl. In an embodiment the alkyl is a C₂-C₇ alkenyl. In embodiments the alkenyl is a C₂, C₃, C₄, C₅, C₆, C₇, C₈, C₉, or C₁₀ alkenyl. The alkenyl group may be substituted if a substituted alkenyl group is indicated.

The alkenyl group may be substituted if a substituted alkenyl group is indicated.

The term “cycloalkenyl” shall mean cyclic rings of 3 to 10 carbon atoms and at least 1 carbon to carbon double bond (i.e., cyclopropenyl, cyclobutenyl, cyclopenentyl, cyclohexenyl, cycloheptenyl or cycloocentyl).

The term “alkynyl” refers to a hydrocarbon radical straight or branched, containing at least 1 carbon to carbon triple bond, and up to the maximum possible number of non-aromatic carbon-carbon triple bonds may be present. Thus, “C₂-C₆ alkynyl” means an alkynyl radical having 2 or 3 carbon atoms and 1 carbon-carbon triple bond, or having 4 or 5 carbon atoms and up to 2 carbon-carbon triple bonds, or having 6 carbon atoms and up to 3 carbon-carbon triple bonds. Alkynyl groups include ethynyl, propynyl and butynyl. In an embodiment the alkynyl is a C₂-C₇ alkynyl. In embodiments the alkynyl is a C₂, C₃, C₄, C₅, C₆, C₇, C₈, C₉, or C₁₀ alknyl. The alkynyl group may be substituted if a substituted alkynyl group is indicated.

“Alkylene”, “alkenylene” and “alkynylene” shall mean, respectively, a divalent alkane, alkene and alkyne radical, respectively.

As used herein, “aryl” is intended to mean any stable monocyclic, bicyclic or polycyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. In an embodiment the aryl is a substituted or unsubstituted phenyl.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The term “ester” is intended to a mean an organic compound containing the R—O—CO—R′ group.

The term “phosphate” is intended to mean an organic compound containing the R—O—P(O)(OR′)₂ group. In a non-limiting example, each occurrence of R′ may be identical or different. In a non-limiting example, R′ may be an H, alkyl or negative charge.

The term “sulfate” is intended to mean an organic compound containing the RO—SO₂—OR′ group. In a non-limiting example, R′ may be an H or a negative charge.

The term “sulfonic esters” is intended o mean an organic compound containing the R—O—SO₂R′ group.

The alkyl, alkenyl, alkynyl, cycloalkyl, cycloalkenyl, aryl, heteroaryl and heterocyclyl substituents may be unsubstituted or unsubstituted, unless specifically defined otherwise. In a non-limiting example, a C₂-C₆ alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, aryl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different. In a non-limiting example, an aryl group may be substituted by an alkenylene and an —OMe group.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing compounds of the present invention, one of ordinary skill in the art will recognize that the various substituents, i.e. R₁, R₂, etc. are to be chosen in conformity with well-known principles of chemical structure connectivity.

In the compounds used in the method of the present invention, the substituents may be substituted or unsubstituted, unless specifically defined otherwise.

In the compounds used in the method of the present invention, alkyl, heteroalkyl, aryl, heteroaryl, phosphate, sulfate, sulfonic ester, or ester groups can be further substituted by replacing one or more hydrogen atoms with alternative non-hydrogen groups. These include, but are not limited to, halo, hydroxy, mercapto, amino, carboxy, cyano and carbamoyl.

The various R groups attached to the aromatic rings of the compounds disclosed herein may be added to the rings by standard proceudres, for example those set forth in Advanced Organic Chemistry: Part B: Reaction and Synthesis, Francis Carey and Richard Sundberg, (Springer) 5th ed. Edition. (2007), the content of which is hereby incoporated by reference.

The compounds described in the present invention are in racemic form or as individual enantiomers. The enantiomers can be separated using known techniques, such as those described in Pure and Applied Chemistry 69, 1469-1474, (1997) IUPAC.

The instant compounds may be in a salt form. As used herein, a “salt” is salt of the instant compounds which has been modified by making acid or base salts of the compounds. In the case of compounds used for treatment of cancer, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

The compounds described herein are useful, being based on resveratrol (see refs. 1a-1d) as, inter alia, antioxidants, for inhibiting lipid peroxidation of low-density lipoprotein, for inhibition of platelet aggregation, for inhibiting cyclooxygenase-1, for inhibiting inflammation, and for inhibiting malignant cell proliferation. In addition, the compounds are therapeutically useful for inhibiting or treating cardiovascular diseases, for example atherosclerosis (see refs. 1a-1d).

The resveratrol-related compounds of this invention are useful for protection of plants, such as crops, from fungal problems. Such antifungal properties of resveratrol have been described in Korean Patent No. 2006114090 and in Adrian et al. (2006) Oxidative Stress and Disease (Ch. 20—Resveratrol in Health and Disease), CRC Press. The compounds are useful in antifungal compositions.

The compositions of this invention may be administered in various forms, including those detailed herein. As used herein, “treatment” of a cardiovascular disease encompasses inducing inhibition, regression, or stasis/prevention of the disorder. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed. In an embodiment, a composition is provided comprising an amount of the compound effective to treat a disease as specified above and a pharmaceutical carrier.

As used herein, a “pharmaceutical carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutical carrier.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with anti-cancer compounds, or tumor growth inhibiting compounds, or with other compounds also used to treat neurite damage. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the cancer, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U. S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incoporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The instant compounds may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions of the invention can be coated onto stents for temporary or permanent implantation into the cardiovascular system of a subject.

In some embodiments, the compounds of the invention are present in a purity of greater than 70%, 75%, 80%, 85%, 90%, 95%. In embodiments the purity of the compound is 96%, 97%, 98%, 99% or 100%.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as 12C, 13C, or 14C. Furthermore, any compounds containing 13C or 14C may specifically have the structure of any of the compounds disclosed herein.

It will also be noted that any notation of a hydrogen in structures throughout this application, when used without further notation, are intended to represent all isotopes of hydrogen, such as 1H, 2H, or 311. Furthermore, any compounds containing 2H or 3H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

All combinations of the various elements are within the scope of the invention.

The compounds used in the method of the present invention may be prepared by techniques well know in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5th Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

This invention will be better understood by reference to the Experimental Details which follow, but those skilled in the art will readily appreciate that the specific experiments detailed are only illustrative of the invention as described more fully in the claims which follow thereafter.

Discussion

Synthesis

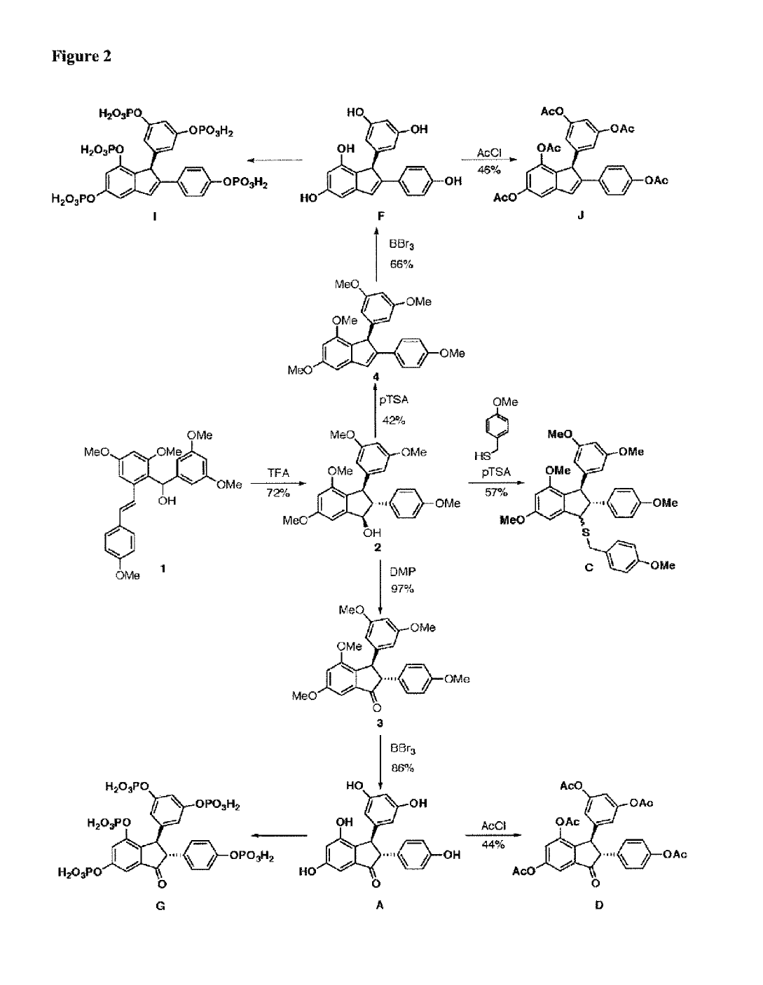

As shown in FIG. 2, when key intermediate 1 was treated with a stoichiometric amount of TFA under carefully controlled conditions (−30→−20° C.) in CH₂Cl₂, a cascade sequence featuring cation generation, regio- and stereoselective cyclization (in relative terms), and stereoselective cation capture, smoothly afforded an intermediate ester after 4 hours. A terminating quench under basic conditions (K₂CO₃, MeOH) then completed the one-pot synthesis of alcohol 2 from 1 in 75% yield. Alcohol oxidation using Dess-Martin periodinane (97% yield) and BBr₃-induced global demethylation in CH₂Cl₂ at 0° C. (86% yield) proceeded smoothly to give polyphneyl A. Polyphenol A was then acetylated with neat acetyl chloride to afford compound D in 44% yield. Elimination using p-toluenesulfonic acid (42% yield) and BBr₃-induced global demethylation in CH₂Cl₂ at 0° C. (66% yield) proceeded to give polyphneyl F. Polyphenol F was then acetylated with neat acetyl chloride to afford compound J in 46% yield. Polyphenols A and F were converted to corresponding phosphates G and I using known methods [5-7]. Exposure of alcohol 2 to p-toluenesulfonic acid for 5 hours followed by addition of p-methoxy-α-toluenethiol at −30° C. afforded sulfide C in 57% yield.

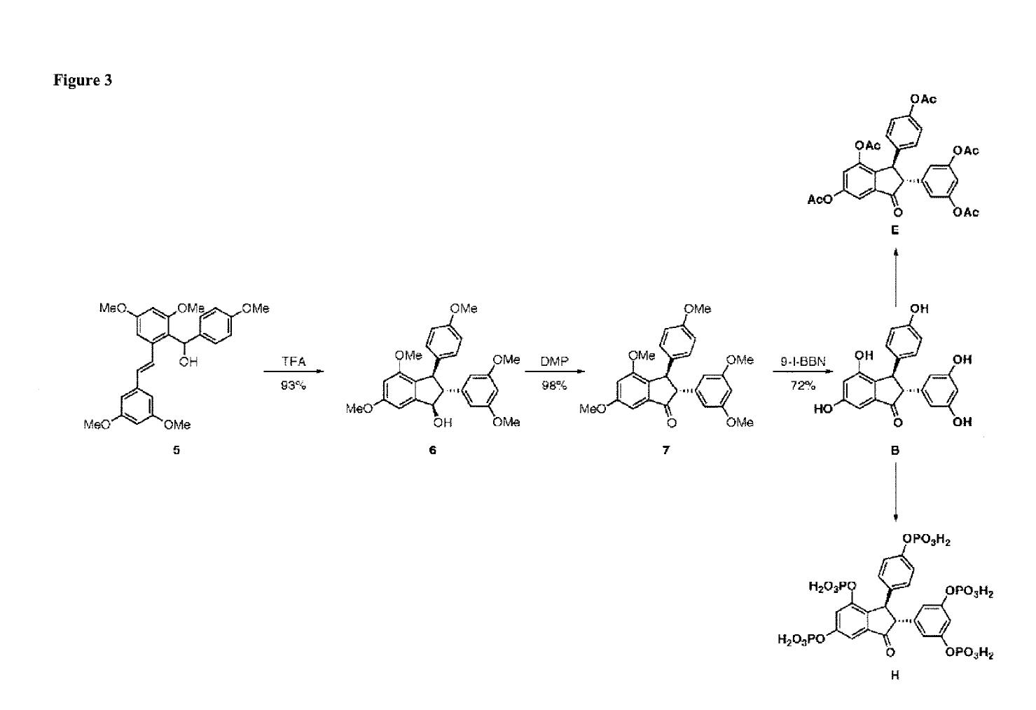

The alternative building block 5 (FIG. 3) behaves in the same manner chemically as alcohol 1. When intermediate 5 was treated with a stoichiometric amount of TFA under carefully controlled conditions (−30→−20° C.) in CH₂Cl₂, a cascade sequence featuring cation generation, regio- and stereoselective cyclization (in relative terms), and stereoselective cation capture, smoothly afforded intermediate ester after 4 hours. A terminating quench under basic conditions (K₂CO₃, MeOH) then completed the one-pot synthesis of alcohol 6 from 5 in 93% yield. Alcohol oxidation using Dess-Martin periodinane (98% yield) and 9-I-BBN-induced global demethylation in CH₂Cl₂ (72% yield) proceeded smoothly to give polyphneyl B. Polyphenol B was then acetylated with neat acetyl chloride to afford compound E in 46% yield. Polyphenol B were converted to corresponding phosphate H using known methods [5-7]

UV Protection

Resveratrol is well documented as a potential sunscreen (see PCT International Publication No. WO 2001/091695 A2, hereby incorporated by reference in its entirety.) In addition, apart from their use as sunscreen agents by blocking UV activity, the known ability of resveratrol to interdict reactive-oxygen species suggests that these analogs are a treatment for various forms of skin cancer.

Fungicidal Activity

Resveratrol and related derivatives have documented fungicidal or antifungicide properties (see PCT International Publication No. WO 2009/038731, hereby incorporated by reference in its entirety.)

Materials and Methods

General Procedures. All reactions were carried out under an argon atmosphere with dry solvents under anhydrous conditions, unless otherwise noted. Dry tetrahydrofuran (THF), acetonitrile (MeCN), toluene, benzene, diethyl ether (Et₂O) and methylene chloride (CH₂Cl₂) were obtained by passing commercially available pre-dried, oxygen-free formulations through activated alumina columns. Yields refer to chromatographically and spectroscopically (¹H and ¹³C NMR) homogeneous materials, unless otherwise stated. Reagents were purchased at the highest commercial quality and used without further purification, unless otherwise stated. Reactions were magnetically stirred and monitored by thin-layer chromatography (TLC) carried out on 0.25 mm E. Merck silica gel plates (60E-254) using UV light as visualizing agent and an ethanolic solution of phosphomolybdic acid and cerium sulfate, and heat as developing agents. SiliCycle silica gel (60, academic grade, particle size 0.040-0.063 mm) was used for flash column chromatography. Preparative thin-layer chromatography (PTLC) separations were carried out on 0.50 mm E. Merck silica gel plates (60E-254). NMR spectra were recorded on Bruker DRX-300, DRX-400 instruments and calibrated using residual undeuterated solvent as an internal reference. The following abbreviations were used to explain the multiplicities: s=singlet, d 32 doublet, t=triplet, br=broad, app=apparent. IR spectra were recorded on a Perkin-Elmer 1000 series FT-IR spectrometer. High-resolution mass spectra (HRMS) were recorded in the Columbia University Mass Spectral Core facility on a JOEL HX110 mass spectrometer using the MALDI (matrix-assisted laser-desorption ionization) technique.

Triaryl Intermediate (1). R_{f}=0.40 (silica gel, EtOAc:hexanes, 1:1); IR (film) vₘₐₓ 3509, 3001, 2938, 2837, 1604, 1511, 1458, 1307, 1244, 1204, 1175, 1153, 1059, 1032, 966, 930, 833, 736 cm⁻¹; ¹H NMR (300 MHz, CDCl₃) δ 7.36 (d, J=8.7 Hz, 2H), 7.28 (d, J=16.2 Hz, 1H), 6.88 (d, J=16.2 Hz, 1H), 6.86 (d, J=8.7 Hz, 2H), 6.74 (d, J=2.1 Hz, 1H), 6.54 (d, J=2.0 Hz, 2H), 6.45 (d, J=2.1, 1H), 6.33 (t, J=2.4, 1H), 6.22 (d, J=9.0 Hz, 1H), 3.86 (s, 3H), 3.80 (s, 3H), 3.78 (s, 1H), 3.74 (s, 6H), 3.72 (s, 3H); ¹³C NMR (75 MHz, CDCl₃) δ 160.5, 159.8, 159.4, 158.6, 147.5, 138.7, 131.5, 129.9, 127.8, 124.4, 121.7, 114.0, 103.8, 103.1, 98.6, 98.3, 70.0, 55.7, 55.3, 55.1; HRMS (FAB) calcd for C₂₆H₂₈O₆⁺ [M⁺] 436.1886, found 436.1870.

Alcohol (2). TFA (1.0 mL, 12.4 mmol, 1.0 equiv) was added in a single portion to a solution of key intermediate 1 (5.4 g, 12/4 mmol, 1.0 equiv) in CH₂Cl₂ (300 mL) at 25°. The resultant dark purple reaction mixture was stirred for 3 min at 25° C. changing color to dark orange. Upon completion, the reaction mixture was quenched sequentially with solid K₂CO₃ (17.1 g, 124 mmol, 10 equiv) and MeOH (140 mL) and stirred for 30 min at 25° C. The reaction contents were then poured into water (40 mL) and extracted with EtOAc (3×400 mL). The combined organic layers were washed with water (60 mL) and brine (60 mL), dried (MgSO₄), and concentrated. The resultant brown oil was purified by flash column chromatography (silica gel, EtOAc:hexanes, 2:1) to give alcohol 2 (4.0 g, 74% yield) as an amorphous white solid. 2: R_{f}=0.41 (silica gel, EtOAc:hexanes, 1:1); IR (film) vₘₐₓ 2935, 1597, 1512, 1463, 1304, 1248, 1203, 1151, 1060, 829 cm⁻¹; ¹H NMR (300 MHz, CDCl₃) δ 7.09 (d, J=8.7 Hz, 2H), 6.83 (d, J=8.7 Hz, 2H), 6.65 (d, J=2.1 Hz, 1H), 6.42 (d, J=2.1 Hz, 1H), 6.27 (t, J=2.3 Hz, 1H), 6.17 (d, J=2.4 Hz, 2H), 5.13 (app t, J=5.7 Hz, 1H), 4.19 (d, J=6.9 Hz, 1H), 3.86 (s, 3H), 3.79 (s, 3H), 3.68 (s, 3H), 3.59 (s, 3H), 3.18 (d, J=6.6 Hz, 1H); ¹³C NMR (75 MHz, CDCl₃) δ 161.7, 160.4, 158.5, 157.1, 146.9, 146.3, 134.0, 128.7, 122.9, 113.9, 105.5, 99.7, 99.4, 99.3, 98.0, 82.5, 66.1, 55.6, 55.3, 55.2, 54.7; FIRMS (FAB) calcd for C₂₆H₂₈O₆⁺ [M⁺] 436.1886, found 436.1870.

Paucifloral F (A). Dess-Martin periodinane (0.152 g, 0.358 mmol, 1.2 equiv) was added in a single portion to a solution of alcohol 2 (0.130 g, 0.298 mmol, 1.0 equiv) in CH₂C₂ (8 mL) at 25° C., and the resultant slurry was stirred for 4 h at 25° C. Upon completion, the reaction contents were quenched with saturated aqueous Na₂SO₃ (10 mL) followed by stirring the resultant biphasic system vigorously for 5 min at 25° C. The reaction contents were then poured into saturated aqueous NaHCO₃ (5 mL) and extracted with EtOAc (3×20 mL). The combined organic layers were washed with water (10 mL) and brine (10 mL), dried (MgSO₄), and concentrated to afford permethylated paucifloral F (0.122 g, 97% yield) as a light yellow oil which was carried forward without additional purification. 3: R_{f}=0.45 (silica gel, EtOAc:hexanes, 1:1); IR (film) vₘₐₓ 1696, 1614, 1514, 1474, 1347, 1155, 1082, 1005, 842 cm⁻¹; ¹H NMR (300 MHz, CDCl₃) δ 7.02 (d, J=8.7 Hz, 2H), 6.90 (d, J=2.1 Hz, 1H), 6.84 (d, J=8.7 Hz, 2H), 6.70 (d, J=2.1 Hz, 1H), 6.32 (app t, J=2.4 Hz, 1H), 6.16 (d, J=2.4 Hz, 2H), 4.44 (d, J=2.7 Hz, 1H), 3.88 (s, 3H), 3.78 (s, 3H), 3.71 (s, 3H), 3.69 (s, 3H), 3.65 (d, J=3.0 Hz, 1H); ^{D}C NMR (75 MHz, CDCl₃) δ 205.9, 162.0 (2 C), 160.8, 158.6, 157.8, 145.9, 138.7, 137.6, 131.5, 128.8, 114.2 (2 C), 106.4, 105.1 (2 C), 98.1, 96.4, 64.1, 55.8, 55.6, 55.2, 51.9. Finally, a solution of this newly synthesized ketone (0.035 g, 0.081 mmol, 1.0 equiv) in CH₂Cl₂ (3 mL) was added dropwise to a commercially-prepared solution of BBr₃ (0.770 mL, 1.0 M in CH₂Cl₂, 0.810 mmol, 10 equiv) at 25° C., and the resultant solution was stirred at 25° C. for 8 h. Upon completion, the reaction mixture was quenched with NaHCO₃ (5 mL), poured into water (10 mL), and extracted with EtOAc (3×20 mL). The combined organic layers were then washed with water (5 mL) and brine (5 mL), dried (MgSO₄), and concentrated. The resultant light pink product was purified by flash column chromatography (silica gel, CH₂Cl₂:MeOH, 9:1) to give paucifloral F (A, 0.025 g, 86% yield) as an amorphous white solid. A: R_{f}=0.06 (silica gel, CH₂Cl₂:MeOH, 9:1); IR (film) vₘₐₓ 3334, 1696, 1614, 1514, 1474, 1347, 1155, 1082, 1005, 842 cm⁻¹; ¹H NMR (300 MHz, Acetone-d₆) δ 8.75 (s, 1H), 8.49 (s, 1H), 8.27 (s, 1H), 8.07 (s, 2H), 6.96 (d, J=8.7 Hz, 2H), 6.78 (d, J=8.7 Hz, 2H), 6.72 (s, 2H), 6.19 (app t, J=2.1 Hz, 1H), 6.02 (d, J=2.1 Hz, 2H), 4.38 (d, J=2.7 Hz, 1H), 3.50 (d, J=2.7, 1H); ¹³C NMR (75 MHz, Acetone-d₆) δ 205.5, 160.2, 159.5, 157.2, 156.7, 147.3, 140.0, 134.8, 131.8, 129.6, 116.3, 110.2, 106.3, 101.6, 100.5, 65.3, 52.1; HRMS (FAB) calcd for C₂₁H₁₇O₆⁺ [M+H^{+]} 365.1025, found 365.1055.

Acetylated Paucifloral F (D). Paucifloral F (A, 0.05 g, 0.13 mmol, 1.0 equiv) was dissolved in neat acetyl chloride (1.25 mL) and stirred at 25° C. for 8 h. The reaction contents were then poured into EtOAc (5 mL) and then quenched with saturated aqueous NaHCO₃ (5 mL). The reaction contents were then extracted with EtOAc (3×10 mL). The combined organic layers were then washed with water (5 mL) and brine (5 mL), dried (MgSO₄), and concentrated. The resultant light yellow product was purified by flash column chromatography (silica gel, EtOAc:hexanes, 1:1) to give acetylated paucifloral F (D) as a white solid (0.042 g, 53%). D: ¹H NMR (400 MHz, CDCl₃) δ 7.52 (d, J=2.0 Hz, 1H), 7.21 (d, J=2.4 Hz, 1H), 7.09-7.04 (m, 4H), 6.84 (t, J=2.0 Hz, 1H), 6.64 (d, J=2.0 Hz, 2H), 4.46 (d, J=5.2 Hz, 1H), 3.74 (d, J=5.2 Hz, 1H), 2.31 (s, 3H), 2.27 (s, 6H), 2.23 (s, 6H), 1.81 (s, 3H); ¹³C NMR (100 MHz, CDCl₃) δ 202.4, 169.4, 168.9, 168.8, 167.9, 151.8, 151.4, 150.1, 148.4, 143.0, 142.4, 138.6, 134.5, 129.5, 123.2, 122.1, 118.2, 114.9, 114.6, 64.4, 52.3, 21.2, 21.0 (2 C), 19.9.

Sulfide (C). p-methoxy-α-toluenethiol (9.6 mL, 68.6 mmol, 3.0 equiv) and p-TsOH (3.96 g, 22.9 mmol, 1.0 equiv) were added to a highly concentrated solution of alcohol 2 (10.0 g, 22.9 mmol, 1.0 equiv) in benzene (2 mL) at 25° C. The resulting yellow-green solution was stirred for 2 h at 25° C. on the rotavap, with periodic addition of benzene to restore the original volume. Upon completion, the reaction mixture was quenched with saturated aqueous NaHCO₃ (30 mL), poured into water (30 mL), and extracted with EtOAc (3×200 mL). The combined organic layers were then washed with water (50 mL) and brine (30 mL), dried (MgSO₄), and concentrated. The resultant light green product was purified by flash column chromatography (silica gel, EtOAc:hexanes, 1:3) to give the sulfide C (10.8 g, 82%) as a light yellow oil. R_{f}=0.71 (silica gel, EtOAc:hexanes, 1:1); IR (film) vₘₐₓ 2995, 2934, 2831, 1607, 1512, 1463, 1421, 1326, 1303, 1249, 1203, 1175, 1154, 1061, 1035, 934, 830 cm⁻¹; ¹H NMR (300 MHz, CDCl₃, 1:1 mixture of diastereomers) δ 7.13 (d, J=8.4 Hz, 2H), 7.07 (d, J=8.7 Hz, 2H), 7.04 (d, J=9.0 Hz, 2 H), 7.03 (d, J=8.4 Hz, 2H), 6.84 (d, J=2.4 Hz, 2H), 6.80 (d, J=2.7 Hz, 2H), 6.79 (s, 1H), 6.77 (s, 1H), 6.74 (d, J=8.7 Hz, 2H), 6.53 (d, J=1.5 Hz, 1H), 6.45 (d, J=1.5 Hz, 1H), 6.36 (br m, 3H), 6.28 (br m, 2H), 6.18 (br m, 4H), 4.55 (s, 1H), 4.53 (d, J=2.7 Hz, 1H), 4.22 (app t, J=7.2 Hz, 3H), 3.82 (s, 3H), 3.81 (s, 3H), 3.80 (s, 3H), 3.79 (s, 3H), 3.77 (s, 3H), 3.76 (s, 3H), 3.69 (s, 3H), 3.68 (s, 6H), 3.61 (s, 3H), 3.57 (s, 6H); ¹³C NMR (75 MHz, CDCl₃, 1:1 mixture of diastereomers) δ 161.5, 161.3, 160.5, 160.3, 158.5, 157.0, 156.8, 147.1, 146.5, 146.2, 145.3, 135.7, 133.5, 130.3, 130.0, 129.8, 128.6, 124.1, 123.7, 113.9, 113.8, 113.7, 113.3, 105.5, 100.8, 100.4, 98.9, 98.5, 98.1, 97.9, 64.6, 60.3, 57.2, 56.7, 55.5, 55.2, 54.0, 53.7, 36.0, 34.9; HRMS (FAB) calcd for C₃4H₃₅O₆S⁺ [M−H⁺] 571.2154, found 571.2168.

Permethylated Alkene (4). To a solution of alcohol 2 (0.100 g, 0.23 mmol, 1.0 equiv) in dry CH₂Cl₂ (2 mL) at 25° C. was added p-TsOH (0.044 g, 0.23 mmol, 1.0 equiv) in one portion. The reaction mixture was allowed to stir at 25° C. for 5 h. Upon completion, the reaction was quenched with saturated aqueous NaHCO₃ (5 mL) and extracted with EtOAc (3×10 mL). The combined organic layers were then washed with water (5 mL) and brine (5 mL), dried (MgSO₄), and concentrated. The resultant yellow oil was purified by flash column chromatography (silica gel, EtOAc:hexanes 1:1) to afford permethylated alkene 4 as a white solid (0.041 g, 42%). 4: ¹H NMR (400 MHz, CDCl₃) δ 7.44 (d, J=9.2 Hz, 2H), 7.06 (d, J=0.9 Hz, 1H), 6.79 (d, J=8.8 Hz, 2H), 6.60 (d, J=2.0 Hz, 1H), 6.40 (d, J=2.0 Hz, 2H), 6.25 (d, J=2.0 Hz, 1H), 6.24 (t, J=2.4 Hz, 1H), 4.93 (d, J=0.9 Hz, 1H), 3.84 (s, 3H), 3.76 (s, 3H), 3.69 (s, 6H), 3.67 (s, 3H); ¹³C NMR (100 MHz, CDCl₃) δ 161.2, 160.4, 159.1, 155.9, 151.7, 146.1, 142.0, 129.2, 128.0 (2 C), 127.9, 125.8, 114.0, 106.7, 98.7, 98.2, 96.4, 55.6 (2 C), 55.2, 54.3.

Alkene (F). A solution of permethylated alkene 4 (0.05 g, 0.12 mmol, 1.0 equiv) in CH₂Cl₂ (7 mL) was added dropwise to a commercially-prepared solution of BBr₃ (0.770 mL, 1.0 M in CH₂Cl₂, 0.810 mmol, 10 equiv) at 25° C., and the resultant solution was allowed stir at 25° C. for 8 h. Upon completion, the reaction mixture was quenched with NaHCO₃ (10 mL), poured into water (20 mL), and extracted with EtOAc (3×20 mL). The combined organic layers were then washed with water (5 mL) and brine (5 mL), dried (MgSO₄), and concentrated. The resultant light pink product was purified by flash column chromatography (silica gel, CH₂Cl₂:MeOH, 9:1) to give alkene F (0.028 g, 66% yield) as an amorphous white solid. F: ¹H NMR (400 MHz, Acetone-d₆) δ 7.45 (d, J=8.8 Hz, 2H), 7.03 (d, J=0.9 Hz, 1H), 6.74 (d, J=8.8 Hz, 2H), 6.45 (d, J=2.0 Hz, 1H), 6.22 (d, J=2.0 Hz, 2H), 6.11 (d, J=2.0 Hz, 1H), 6.08 (t, J=2.0 Hz, 1H), 4.89 (d, J=0.9 Hz, 1H); ¹³C NMR (100 MHz, Acetone-d₆) δ 159.1, 159.0, 157.8, 153.9, 152.8, 147.6, 143.3, 128.9, 128.0, 125.8, 125.7, 116.0, 107.7, 101.6, 101.2, 100.9, 54.3.

Acetylated Alkene (J). Alkene F (0.035 g, 0.09 mmol, 1.0 equiv) was dissolved in neat acetyl chloride (1 mL) and stirred at 25° C. for 8 h. The reaction contents were then poured into EtOAc (5 mL) and then quenched with saturated aqueous NaHCO₃ (5 mL). The reaction contents were then extracted with EtOAc (3×10 mL). The combined organic layers were then washed with water (5 mL) and brine (5 mL), dried (MgSO₄), and concentrated. The resultant light yellow product was purified by flash column chromatography (silica gel, EtOAc:hexanes, 1:1) to give acetylated alkene (J) as a white solid (0.028 g, 50%). J: ¹H NMR (400 MHz, CDCl₃) δ 7.42 (d, J=8.4 Hz, 2H), 7.11 (d, J=0.9 Hz, 1H), 7.07 (d, J=2.0 Hz, 1H), 7.00 (d, J=8.7 Hz, 2H), 6.82 (t, J=2.0 Hz, 1H), 6.71 (d, J=2.0 Hz, 2H), 6.67 (d, J=2.0 Hz, 1H), 4.92 (d, J=0.9 Hz, 1H), 2.29 (s, 3H), 2.26 (s, 3H), 2.21 (s, 6H), 2.08 (s, 3H); ¹³C NMR (100 MHz, CDCl₃) δ 169.4, 169.3, 168.7, 168.4, 151.5, 151.3, 150.6, 146.5, 146.3, 139.9, 135.9, 132.0, 128.0, 127.4, 121.9, 118.6, 114.3, 113.1, 112.7, 54.5, 31.1, 21.3 (2 C), 21.2, 20.6.

Triaryl Intermediate (5) R_{f}=0.45 (silica gel, EtOAc:hexanes, 1:1); IR (film) vₘₐₓ 3508, 3001, 2938, 2837, 1599, 1510, 1459, 1425, 1323, 1283, 1246, 1203, 1152, 1064, 1035, 964, 835, 799, 736 cm⁻¹; ¹H NMR (300 MHz, CDCl₃) δ 7.36 (d, J=15.9 Hz, 1H), 7.24 (d, J=8.4 Hz, 2H), 6.84 (d, J=15.9 Hz, 1H), 6.82 (d, J=8.7 Hz, 2H), 6.74 (d, J=2.4 Hz, 1H), 6.56 (d, J=2.1 Hz, 2H), 6.48 (d, J=2.4 Hz, 1H), 6.38 (t, J=2.1 Hz, 1H), 6.23 (d, J=9.9 Hz, 1H), 3.87 (s, 3H), 3.80 (s, 6H), 3.77 (s, 3H), 3.73 (s, 3H); ¹³C NMR (75 MHz, CDCl₃) 6 160.9, 159.8, 158.8, 158.3, 139.1, 138.2, 136.8, 132.0, 127.1, 126.9, 122.3, 113.4, 104.6, 103.3, 100.3, 99.1, 69.8, 55.7, 55.4, 55.3, 55.2; HRMS (FAB) calcd for C₂₆H₂₈O₆⁺ [M⁺] 436.1886, found 436.1870.

Isopaucifloral F (B) was synthesized from intermediate 5 exactly as described above for paucifloral F (A). Only the final deprotection leading to isopaucifloral F (B) is fundamentally different from the steps outlined above, so only this procedure is defined specifically below.

Alcohol (6). ¹H NMR (300 MHz, CDCl₃) δ 6.97 (d, J=8.7 Hz, 2H), 6.93 (d, J=8.7 Hz, 2H), 6.73 (d, J=1.8 Hz, 1H), 6.41 (d, J=1.8 Hz, 1H), 6.34-6.31 (m, 3H), 5.17 (d, J=6.6 Hz, 1H), 4.25 (d, J=7.2 Hz, 1H), 3.86 (s, 3H), 3.76 (s, 3H), 3.73 (s, 6H), 3.53 (s, 3H), 3.15 (t, J=6.9 Hz, 1H).

Permethylated isopaucifloral (7). ¹H NMR (300 MHz, CDCl₃) δ 6.94 (d, J=8.5 Hz, 2H), 6.90 (d, J=1.8 Hz, 1H), 6.78 (d, J=8.7 Hz, 2H), 6.69 (d, J=1.8 Hz, 1H), 6.35 (t, J=2.0 Hz, 1H), 6.24 (d, J=2.0 Hz, 2H), 4.51 (d, J=2.6 Hz, 1H), 3.61 (d, J=2.6 Hz, 1H), 3.87 (s, 3H), 3.77 (s, 3H), 3.73 (s, 6H), 3.65 (s, 3H).

Isopaucifloral F (B). 9-I-BBN (1.61 mL, 1.0 M in hexanes, 1.61 mmol, 7.0 equiv) was added dropwise to a solution of permethylated isopaucifloral F (7) (0.100 g, 0.240 mmol, 1.0 equiv) in CH₂Cl₂ (10 mL) at 25° C. The reaction solution turned a red color immediately, and was immediately heated at 40° C. for 8 h with continued stirring. Upon completion, the reaction mixture was cooled to 25° C., quenched with water (15 mL), and extracted with EtOAc (3×20 mL). The combined organic layers were then washed with water (15 mL) and brine (15 mL), dried (MgSO₄), and concentrated. The resultant red oil was purified by flash column chromatography (silica gel, CH₂Cl₂:MeOH, 9:1) to afford isopaucifloral F (B, 0.063 g, 72%) as colorless oil. B: R_{f}=0.06 (silica gel, CH₂Cl₂:MeOH, 9:1); IR (film) vₘₐₓ 3349, 1691, 1602, 1512, 1418, 1342, 1251, 1149 cm⁻¹; ¹H NMR (300 MHz, Acetone-d₆) δ 8.13 (s, 3H), 7.35 (s, 2H), 6.89 (d, J=8.7 Hz, 2H), 6.74 (d, J=8.7 Hz, 2H), 6.71 (d, J=2.1 Hz, 1H), 6.24 (t, J=2.1 Hz, 1H), 6.11 (d, J=2.1 Hz, 2H), 4.48 (d, J=2.4 Hz, 1H), 3.42 (d, J=2.7 Hz, 1H); ¹³C NMR (75 MHz, Acetone-d₆) δ 205.1, 160.2, 159.6, 156.8, 156.6, 143.3, 140.1, 135.7, 135.3, 128.9, 116.1, 110.3, 107.0, 102.1, 100.7, 66.3, 51.4; HRMS (FAB) calcd for C₂₁H₆O₆⁺ [M⁺] 364.0947, found 364.0961.

Acetylated Isopaucifloral F (E). Isopaucifloral F (B, 0.061 g, 0.16 mmol, 1.0 equiv) was dissolved in neat acetyl chloride (1.5 mL) and stirred 25° C. for 8 h. The reaction contents were then poured in EtOAc (4 mL) and then quenched with saturated aqueous NaHCO₃ (10 mL). The reaction contents were then extracted with EtOAc (3×20 mL). The combined organic layers were then washed with water (10 mL) and brine (10 mL), dried (MgSO₄), and concentrated. The resultant light yellow product was purified by flash column chromatography (silica gel, EtOAc/Hex, 1:1) to give acetylated isopaucifloral F as an amorphous white solid (E, 0.055 g, 54%). E: 1H NMR (400 MHz, CDCl₃) δ 7.53 (d, J=2.0 Hz, 1H), 7.19 (d, J=2.0 Hz, 1H), 7.08-7.02 (m, 4H), 6.93 (t, =2.0 Hz, 1H), 6.74 (d, =2.0 Hz, 2H), 4.50 (d, J=5.2 Hz, 1H), 3.75 (d, J=5.2 Hz, 1H), 2.33 (s, 3H), 2.29 (s, 3H), 2.25 (s, 6H), 1.78 (s, 3H); ¹³C NMR (100 MHz, CDCl₃) δ 201.9, 169.5, 168.9, 168.8, 167.8, 151.4, 150.1, 148.5, 143.1, 139.4, 138.6, 138.1, 129.2, 128.7, 128.4, 123.5, 122.4, 119.0, 114.8, 114.7, 64.5, 51.9, 21.2 (2 C), 20.0.

Phosphorylated materials are to be prepared using generalized procedures found in the literature for phenol derivatization, for which numerous protocols are known. For example, one could envision use of POCl₃, potentially in the presence of additional acidic or basic species, to afford these materials directly in an appropriate solvent following an appropriate work-up. Alternatively, one could envision the initial use of a dialkyl phosphite (such as dibenzyl phosphite) in an appropriate solvent, potentially in the presence of additional acidic or basic species, to generate an intermediate protected phosphonate species that can then be deprotected to give the desired phosphonate either through hydrolysis or alkyl ether cleavage under appropriate conditions. In the case of benzyl ethers, conditions that could be used, among others, would include TMSBr in an appropriate solvent followed by water addition or hydrogenation over appropraite metal catalysts such as Pd/C in appropriate solvents. The generalized as described in Chen et al. [5], Mills et al. [6], and PCT International Application No. WO 2006/029484 [7], would be an example of such a procedure to prepare phosphorylated materials, including the demonstration of phosphorylating the three phenol residues of resveratrol. As an alternative to such a procedure, one could also envision the use of a dialkylphosphoryl chloride [such as dibenzylphosphoryl chloride, also known as phosphorochloridic acid, bis(phenylmethyl) ester], either neat or in an appropriate solvent, potentially in the presence of additional acidic or basic species, to form the same types of initial protected phosphonate species from the starting phenol prior to their subsequent conversion to the desired phosphonate.

### REFERENCES

[1] a) M. Jang, L. Cai, G. O. Udeani, K. V. Slowing, C. F. Thomas, C. W. W. Beecher, H. H. S. Fong, N. R. Farnsworth, A. D. Kinghorn, R. G. Mehta, R. C. Moon, J. M. Pezzuto, Science 1997, 275, 218-220; b) K. T. Howitz, K. J. Bitterman, H. Y. Cohen, D. W. Lamming, S. Lavu, J. G. Wood, R. E. Zipkin, P. Chung, A. Kisielewski, L.-L. Zhang, B. Scherer, D. A. Sinclair, Nature 2003, 425, 191-196; c) L. M. Szewczuk, L Forti, L A. Stivala, T. M. Penning, J. Biol. Chem. 2004, 21, 22727-22737; d) J. A. Baur, K. J. Pearson, N. L. Price, H. A. Jamieson, C. Lerin, A. Kalra, V. V. Prabhu, J. S. Allard, G. Lopez-Lluch, K. Lewis, P. J. Pistell, S. Poosala, K. G. Becker, O. Boss, D. Gwinn, M. Wang, S. Ramswamy, K. W. Fishbein, R. G. Spencer, E. G. Lakatta, D. Le Couteur, R. J. Shaw, P. Navas, P. Puigserver, D. K. Ingram, R. de Cabo, D. A. Sinclair, Nature 2006, 444, 337-342.

[2] G. J. Soleas, E. P. Diamandis, D. M. Goldberg, Clin. Biochemistry 1997, 30, 91-113.

[3] a) Y. Oshima, Y. Ueno, K. Hisamachi, M. Takeshita, Tetrahedron 1993, 49, 5801-5804; b) Y. Oshima, Y. Ueno, Phytochemistry 1993, 33, 179-182; c) M. Niwa, J. Ito, K. Terashima, T. Koizumi, Y. Takaya, K.-X. Yan, Heterocycles 2000, 53, 1475-1478; d) J. Ito, T. Tanaka, M. Iinuma, K. Nakaya, Y. Takahashi, R. Sawa, J. Murata, D. Darnaedi, J. Nat. Prod. 2004, 67, 932-937; e) H.-F. Luo, L.-P. Zhang, C.-Q. Hu, Tetrahedron 2001, 57, 4849-4854; f) M. A. Khan, S. G. Nabi, S. Prakash, A. Zaman, Phytochemistry 1986, 25, 1945-1948; g) H. A. Guebailia, K. Chira, T. Richard, T. Mabrouk, A. Furiga, X. Vitrac, J.-P. Monti, J.-C. Delaunay, J.-M. Merillon, J. Agric. Food Chem. 2006, 54, 9559-9564; h) T. Tanaka, T. Ito, K. Nakaya, M. Iinuma, S. Riswan, Phytochemistry 2000, 54, 63-69; i) Y. Takaya, K.-X. Yan, K. Terashima, J. Ito, M. Niwa, Tetrahedron 2002, 58, 7259-7265; j) B. Supudompol, K. Likhitwitayawuid, P. J. Houghton, Phytochemistry 2004, 65, 2589-2594; k) N. S. Aminah, S. A. Achmad, N. Aimi, E. L. Ghisalberti, E. H. Hakim, M. Kitajima, Y. M. Syah, H. Takayama, Filoterapia 2002, 73, 501-507.

[4] a) M. Ohyama, T. Tanaka, T. Ito, M. Iinuma, K. F. Bastow, K.-H. Lee, Bioorg. Med. Chem. Lett. 1999, 9, 3057-3060; b) K. Ohguchi, T. Tanaka, T. Ito, M. Iinuma, K. Matsumoto, Y. Akao, Y. Nozawa, Biosci. Biotechnol. Biochem. 2003, 67, 1587-1589; c) K. Ohguchi, Y. Akao, K. Matsumoto, T. Tanaka, T. [to, M. Iinuma, Y. Nozawa, Biosci. Biotechnol. Biochem. 2005, 69, 353-356; d) T. Ito, Y. Akao, H. Yi, K. Ohguchi, K. Matsumoto, T. Tanaka, M. Iinuma, Y. Nozawa, Carcinogenesis 2003, 24, 1489-1497.

[5] K. Chen, A. Aowad, S. Adelstein, and A. Kassis, J. Med. Chem. 2007, 50, 663-673.

[6] S. Mills, H. Dozol, F. Vandeput, K. Backers, T. Woodman, C. Erneux, B. Spiess, and B. Potter ChemBioChem 2006, 7, 1696-1706.

[7] PCT International Publication No. WO 2006/029484 A1, published Mar. 23, 2006.

1. A compound having the structure: 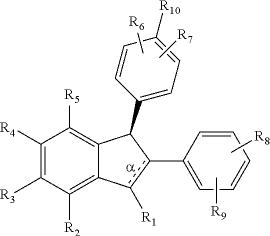
wherein
bond α is present or absent,
wherein when bond α is absent, then R₁ is ═O or S(CH₂)₁₋₃-phenyl and when bond a is present, then R₁ is H,
wherein the phenyl is substituted or unsubstituted;
R₂, R₃, R₄, R₅, R₆, R₇, R₈, R₉ and R₁₀ are independently H or OR₁₁
wherein each occurrence of R₁₁ is independently H, methyl, substituted or unsubstituted alkyl, substituted or unsubstituted heteroalkyl substituted or unsubstituted aryl, substituted or unsubstituted heteroaryl, phosphate, sulfate, sulfonic ester, or ester;
when α is absent and R₁ is ═O, then R₁₀ is other than OH; and
when α is absent and R₁ is S(CH₂)₁₋₃-phenyl, then R₁₀ is other than OCH₃,
or a salt thereof.

2. The compound of claim 1,
wherein each occurrence of R₁₁ is independently H, CH₃, C(O)CH₃, P(O)(OR₁₂)₂, SO₂OR₁₂, SO₂R₁₃, or C(O)R₁₃,
wherein each occurrence of R₁₂ is independently H, methyl, substituted or unsubstituted alkyl, or substituted or unsubstituted aryl,
wherein each occurrence of R₁₃ is independently H, methyl, substituted or unsubstituted alkyl, or substituted or unsubstituted aryl,
or a salt thereof.

3. The compound of claim 2,
wherein
R₃ and R₅ are OR₁₁; and
R₂ and R₄ are H,
or a salt thereof.

4. The compound of claim 1,
wherein the structure is 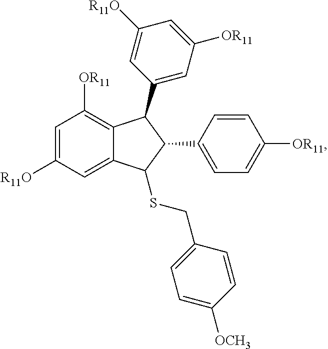
or a salt thereof.

5. The compound of claim 4,
wherein R₁₁ is H, CH₃, C(O)CH₃, or P(O)(OR₁₂)₂,
wherein R₁₂ is H,
or a salt thereof.

6. The compound of claim 5,
wherein the structure is 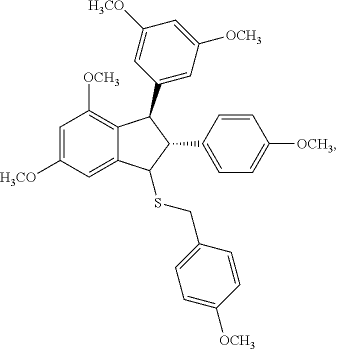 [SMILES: COc1ccc(CSC2c3cc(OC)cc(OC)c3[C@@H](c3cc(OC)cc(OC)c3)[C@@H]2c2ccc(OC)cc2)cc1]
or a salt thereof.

7. The compound of claim 1,
wherein the structure is 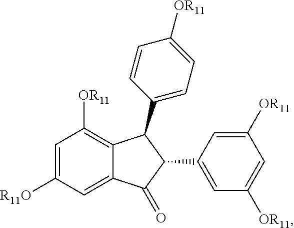
or a salt thereof.

8. The compound of claim 7,
wherein R₁₁ is CH, C(O)CH₃, or P(O)(OR₁₂)₂,
wherein R₁₂ is H,
or a salt thereof.

9. The compound of claim 8,
wherein the structure is 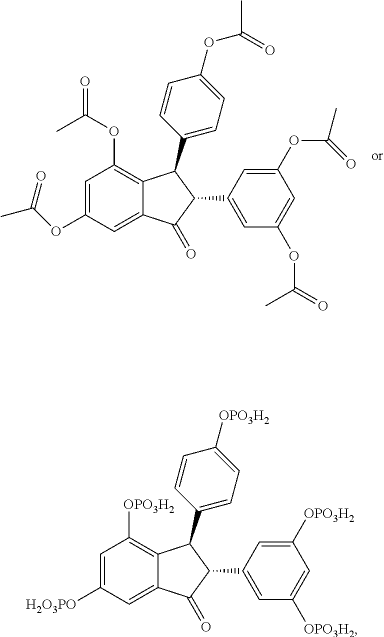
or a salt thereof.

10. The compound of claim 1,
wherein the structure is 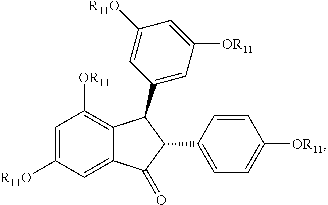
or a salt thereof.

11. The compound of claim 10,
wherein R₁₁ is H, CH₃, C(O)CH;, P(O)(OR₁₂)₂,
wherein R₁₂ is H,
or a salt thereof.

12. The compound of claim 11,
wherein the structure is 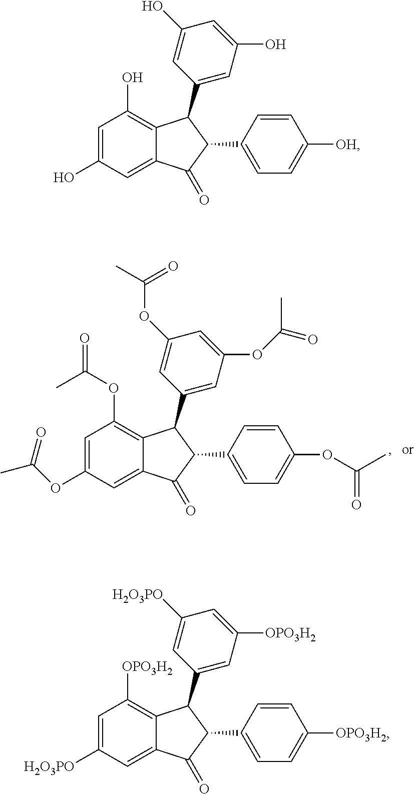
or a salt thereof.

13. The compound of claim 1,
wherein the structure is 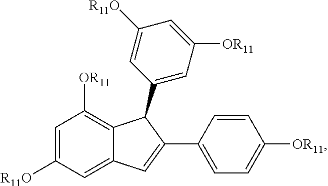
or a salt thereof.

14. The compound of claim 13
wherein R₁₁ is H, CH₃, C(O)CH₃, R(O)(OR₁₂)₂,
wherein R₁₂ is H,
or a salt thereof.

15. The compound of claim 14,
wherein the structure is 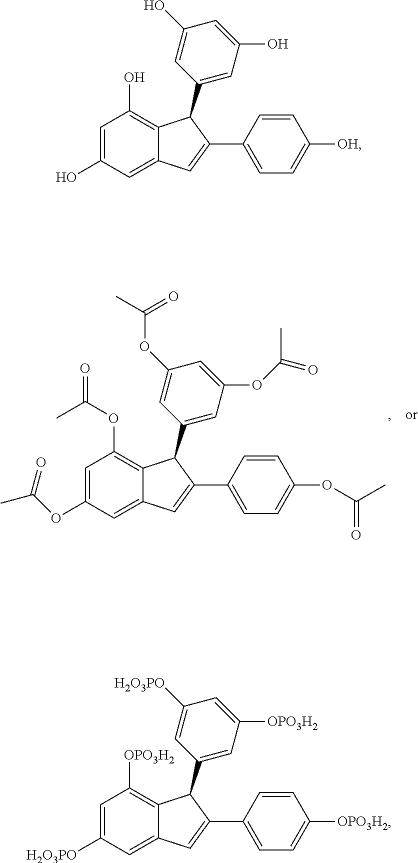
or a salt thereof.

16. The compound of claim 2,
wherein the structure is 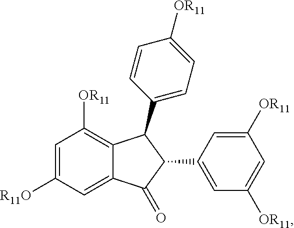
or a salt thereof.

17. The compound of claim 2,
wherein the structure is 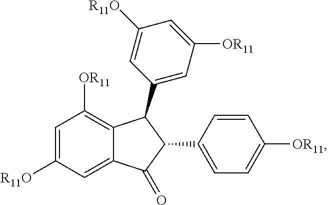
or a salt thereof.

18. The compound of claim 2,
wherein the structure is 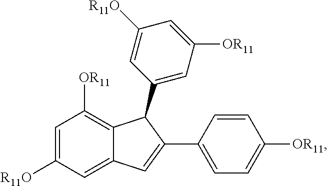
or a salt thereof.

19. The compound of claim 3,
wherein the structure is 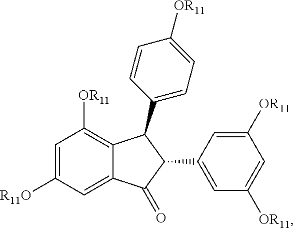
or a salt thereof.

20. The compound of claim 3,
wherein the structure is 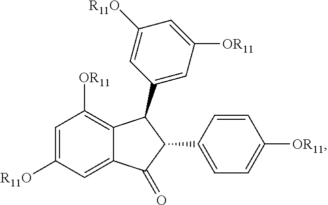
or a salt thereof.
